(19) United States
(12) Plant Patent Application Publication
(10) Pub. No.: US 2009/0038040 P1
(43) Pub. Date: Feb. 5, 2009

(54) Hibiscus 'Panama Red'

(76) Inventor: John Ruter, Tifton, GA (US)

Correspondence Address: DICKINSON WRIGHT PLLC, 38525 WOODWARD AVENUE, SUITE 2000, BLOOMFIELD HILLS, MI, 48304-2970, US

(21) Appl. No.: 11/881,889

(22) Filed: Jul. 30, 2007

(51) Int. Cl. A01H 5/00

(52) U.S. Cl. PLT/257

(57) ABSTRACT

Hibiscus ‘Panama Red’ is an ornamental variety, crossed and cultivated in Georgia. The plants of the new Hibiscus display an intense red color in high light, deeply cut foliage, stable foliage color, very large flowers, thrives in hot an humid conditions, and flowers heavily during short days (November to April) in zone 10. ‘Panama Red’ can be grown in the garden or in a container.

### CROSS-REFERENCE TO RELATED APPLICATIONS

This patent application is related to co-pending application entitled Hibiscus ‘Panama Bronze,’ invented by the same Inventor, assigned to the same Assignee, and filed simultaneously with, the present application. The present application Hibiscus ‘Panama Red’ is the maternal parent plant of cross-referenced Hibiscus ‘Panama Bronze.’

### LATIN NAME

Hibiscus ‘Panama Red’ is of the genus and species Hibiscus acetosella.

### VARIETY DENOMINATION

The new Hibiscus claimed is of the variety denominated ‘Panama Red.’

### BACKGROUND OF THE INVENTION

The present invention relates to a new and distinct cultivar of Hibiscus, botanically known as Hibiscus acetosella, and herein referred to as ‘Panama Red.’

The new Hibiscus is a product of a planned breeding program conducted by the Inventor at the Tifton campus of the University of Georgia in Tifton, Ga. The objective of the Hibiscus breeding program is to create new plant cultivars with ornamental leaf distinctions and thrive in hot and humid conditions.

The new Hibiscus originated from a discovery of a seedling made by the Inventor in 2004, of an unnamed and unpatented red selection of the Hibiscus acetosella.

Asexual reproduction of the new Hibiscus by vegetative terminal cuttings in a controlled environment in Tifton, Ga. since 2004, has shown that the unique features of this new Hibiscus are stable and reproduced true to type in successive generations.

### BRIEF SUMMARY OF THE INVENTION

The cultivar ‘Panama Red’ has not been observed under all possible environmental conditions. The phenotype may vary somewhat with variations in environment and cultural practices such as temperature and light intensity without, however, any variance in genotype.

The following traits have been repeatedly observed and are determine to be the unique characteristics of ‘Panama Red:’
1. Rounded growth habit;
2. Dissect leaves;
3. Stable foliage color;
4. Rich dark red foliage in low light;
5. Blackish-purple foliage in high light;
6. Flowers heavily under short days (November through April) in zone 10; and
7. Thrives in hot and humid conditions.

Plants of the new Hibiscus can be compared to its parents, Hibiscus acetosella, an example of which is known as Hibiscus ‘Red Shield.’ Plants of the new Hibiscus differ from ‘Red Shield’ in the following characteristics:
1. Plants of the new Hibiscus a nearly blackish-purple in high light and a rich, dark red in low light compared to a less intense red in ‘Red Shield.’
2. Plants of the new Hibiscus have a more rounded growth habit compared to ‘Red Shield.’
2. Plants of the new Hibiscus have more dissect leaves compared to ‘Red Shield.’
3. Plants of the new Hibiscus have more petiole barbs compared to ‘Red Shield.’

Plants of the new Hibiscus can be compared to its progeny, ‘Panama Bronze’ (co-pending patent application). Plants of the new Hibiscus differ from ‘Panama Bronze’ in the following characteristics:
1. Plants of the new Hibiscus display a nearly blackish-purple in high light and a rich, dark red in low light compared to ‘Panama Bronze,” which shows an intense bronze color in high light and a green color in low light.
2. Plants of the new Hibiscus have flowers that are one third smaller compared to ‘Panama Bronze.’
3. Plants of the new Hibiscus have less dissect leaves than ‘Panama Bronze.’
4. Plants of the new Hibiscus are more dense compared to ‘Panama Bronze.’

### BRIEF DESCRIPTION OF THE DRAWINGS

FIG. 1 is a photograph of ‘Panama Red’ showing the rounded growth habit.

FIG. 2 is a photograph of a leaf of ‘Panama Red’ showing foliage color and dissect characteristics.

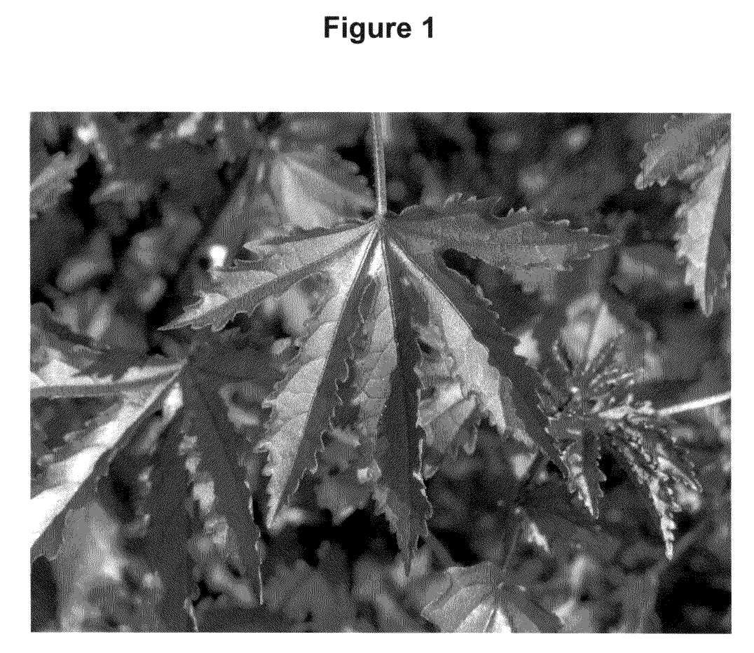

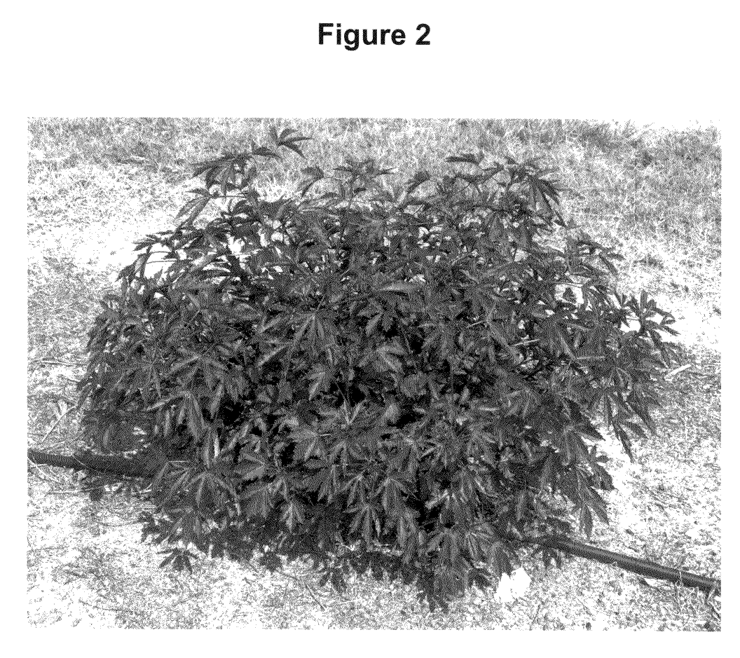

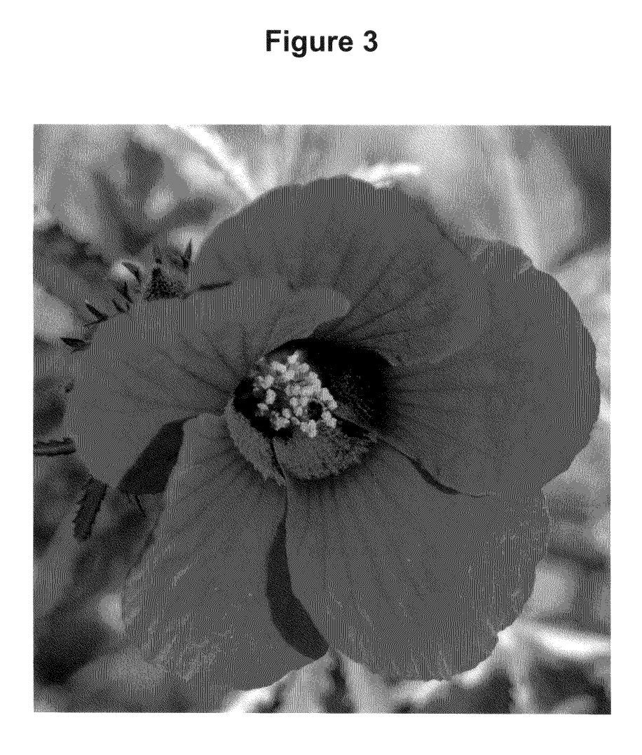

### DETAILED BOTANICAL DESCRIPTION

The following observations, measurements and values describe plants grown in Athens, Ga. at the Athens Select® Test Garden at the University of Georgia Athens campus, by Allan Armitage and Stephanie Anderson. During the growing of the plants, day temperatures ranges from 50° F. to 100+° F. and night temperatures ranges from 35° F. to 80° F. In the description, color references are made to The Royal Horticultural Society (R.H.S.) Colour Chart, 1995, Edition, except where general terms of ordinary dictionary meaning are used.
I. Habit: Round to upright
II. Size of plant:
   A. Height.—5-6′ (1.5-1.8 m).
   B. Width.—2-3′ (0.6-0.9 m).
III. Stem:
   A. Color (R.H.S.).—60A.
   B. Length.—3-4′ (0.9-1.2 m).
   C. Diameter.—3-5 mm.
   D. Pubescence.—none.
   E. Shape.—round.
   F. Odor (of bruised stem).—none.
   G. Internode length.—4-5 cm.
IV. Leaf:
   A. Color (R.H.S.).—1. upper 187B 2. lower 187C.
   B. Mature size (L×W).—9.5×12 cm.
   C. Apex.—acuminate.
   D. Base.—truncate.
   E. Margin.—crenate.
   F. Shape.—palmate.
   G. Lobes (present/absent).—1. number 3-5.
   H. Pubescence.—none.
   I. Arrangement on stem.—alternate.
   J. Venation.—pinnipalmate.
   K. Texture.—glabrous.
Petiole:
   A. Length.—6-7 cm.
   B. Shape.—round.
   C. Color (R.H.S.).—187A.
   D. Pubescence.—none.
   E. Diameter.—0.3 cm.
VI. Flower:
   A. Inflorescence.—none, solitary 1. number of individual flowers per stem 5-10.
   B. Individual flower.—1. axillary, terminal axillary 2. symmetry radial 3. petals 5 a. size (L×W) 5.25×4 cm b. shape spatulate c. apex rounded d. base truncate e. margin entire f. color at peak of bloom 1) upper 187C 2) center 187A 4. pedicels a. color (R.H.S.) 187C b. pubescence none c. length 1-2 mm 5. sepals a. number 5 b. size (L×W) 1.8×0.7 c. shape linear d. pubescence none e. color (R.H.S.) 144B 6. stamens a. number 23-25 b. size (L×W) 0.4×0.8 cm c. color (R.H.S.) 184B d. pollen color (R.H.S.) e. pubescence none 7. pistils a. number 1 b. size of style (L×W) 2.5×0.3 cm c. color of style (R.H.S.) 185B d. color of stigma (R.H.S.) 187A 8. bracts a. number 9-11 b. size (L×W) 1.8×0.5 cm c. color (R.H.S.) 126A d. shape spatulate.
C. Fruit: no fruit seen
D. Seed: no seed seen

What is claimed is:

1. A new and distinct variety of Hibiscus plant named ‘Panama Red’, substantially as illustrated and described herein.
